(19) United States
(12) Patent Application Publication
(10) Pub. No.: US 2006/0270611 A1
(43) Pub. Date: Nov. 30, 2006

(54) PEDIATRIC FORMULATION OF TOPIRAMATE

(76) Inventors: Willy Maria Albert Carlo Dries, Merksplas (BE); Roger Carolus Augusta Embrechts, Oud-Turnhout (BE); Marc Karel Jozef Francois, Kapellen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/419,834

(22) Filed: May 23, 2006

Related U.S. Application Data
(60) Provisional application No. 60/690,391, filed on Jun. 14, 2005.

(30) Foreign Application Priority Data
May 25, 2005 (EP) 05104491.5

(51) Int. Cl. A61K 31/7024

(52) U.S. Cl. 514/23

(57) ABSTRACT

The present invention concerns a liquid preconcentrate composition comprising topiramate or a pharmaceutically acceptable addition salt thereof as active ingredient and an organic solvent, said composition having a low water content; a liquid composition for oral administration obtainable by mixing the composition with an aqueous medium; and processes for preparing the same.

This application claims priority from European application number 05104491.5, filed on May 25, 2005, and U.S. provisional application Ser. No. 60/690,391, filed Jun. 14, 2005, the contents of both of which are hereby incorporated by reference in their entirety.

The present invention concerns a liquid preconcentrate composition comprising topiramate or a pharmaceutically acceptable addition salt thereof as active ingredient and an organic solvent, said composition having a low water content; a liquid composition for oral administration obtainable by mixing the preconcentrate composition with an aqueous medium; and processes for preparing the same.

Topiramate, chemically 2,3:4,5-bis-O-(1-methylethylidene) β-D-fructopyranose sulfamate is represented by the following structure

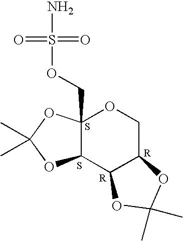

Topiramate has been demonstrated in clinical trials of human epilepsy to be effective as adjunctive therapy or as monotherapy in treating simple and complex partial seizures and secondarily generalized seizures (E. FAUGHT, B. J. WILDER, R. E. RAMSEY, R. A. REIFE, L D. KRAMER, G. W. PLEDGER, R. M. KARIM et. al., Epilepsia 1995, 36 (S4), 33; S. K. SACHDEO, R. C. SACHDEO, R. A. REIFE, P. LIM and G. PLEDGER, Epilepsia 1995, 36 (S4), 33; T. A. GLAUSER, Epilepsia 1999, 40 (S5), S71-80; R. C. SACHDEO, Clin. Pharmacokinet. 1998, 34, 335-346), and is currently marketed for the treatment of seizures in patients with simple and complex partial epilepsy, primary or secondary generalized seizures and seizures associated with Lennox-Gastaut syndrome in the United States, Europe and select other markets throughout the world.

More recently, topiramate has been approved and is currently marketed for the prophylaxis of migraine headaches in adults in the United States, Europe and select other markets throughout the world.

U.S. Pat. No. 4,513,006, which is hereby incorporated by reference, discloses a class of novel anti-epileptic compounds, including topiramate. In addition, U.S. Pat. Nos. 4,513,006 and 5,387,700, which are hereby incorporated by reference, disclose processes for making topiramate and related compounds.

US2003/0077227 relates to buccal spray compositions and soft bite gelatin capsules for transmucosal administration of biologically active compounds.

WO02/102369 describes the use of topiramate for the protection of retinal neurons. It discloses aqueous solutions for injection or as eye drop.

WO2005/048981 relates to a controlled release formulation of topiramate in a liquid dosage form.

The present invention concerns an oral liquid topiramate formulation suitable for pediatric use.

Because of the ease of administration, oral liquid formulations are very suited for pediatric use compared to solid dosage forms, such as tablets or capsules.

Topiramate however is sensitive to hydrolysis in an aqueous medium and therefore can not be formulated as a conventional aqueous solution for oral use. Such an aqueous solution would have a very limited shelf life.

By formulating topiramate as a liquid non-aqueous preconcentrate composition, in particular a liquid preconcentrate composition with a low water content, more in particular a liquid preconcentrate composition in an essentially organic solvent, a formulation can be provided with an acceptable shelf life.

Given the sensitivity of topiramate to hydrolysis in an aqueous medium, the preconcentrate composition has a low water content. A low water content in this context means that the concentration of water in the composition is preferably about 5% by weight or less, more preferably 2.5% by weight or less, even more preferably about 1% by weight or less or that the composition is substantially free of water.

Substantially free of water in this context means that the concentration of water in the composition is preferably about 0.3% by weight or less, even more preferably about 0.2% by weight or less.

Therefore, the present invention relates to a liquid preconcentrate composition comprising topiramate or a pharmaceutically acceptable addition salt thereof, as active ingredient and an organic solvent, said composition having a low water content.

The term “preconcentrate” as used herein is meant to represent a concentrated formulation which has to be diluted before use, preferably with an aqueous medium, e.g. the preconcentrate can be diluted with an aqueous medium when it is dispensed at a pharmacy.

In a preferred embodiment of the present invention, the liquid preconcentrate composition is a solution. The organic solvent must provide sufficient stability and solubility for the active ingredient and for additional ingredients which may be present in the composition.

A liquid preconcentrate solution has the advantage compared to a solid preconcentrate that, in order to form a liquid solution upon reconstitution with an aqueous medium, sufficient mixing suffices whereas for a solid preconcentrate care has to be taken that the solid preconcentrate is completely dissolved upon reconstitution or in case the solid preconcentrate forms a suspension upon reconstitution, that the resulting suspension stays evenly dispersed or that it is easily dispersable upon shaking.

Since the formulation of the present invention is intended for pediatric use, the organic solvent of the preconcentrate composition is preferably an organic solvent suitable for pediatric use. Examples of such organic solvents are ethanol; glycerol; PEG (polyethylene glycol), such as for instance PEG 300, PEG 400, PEG 500 or PEG 600, in particular PEG 300, PEG 400 or PEG 600, more in particular PEG 300 or PEG 400; propylene glycol; or mixtures thereof.

Thus, in a preferred embodiment, the present invention relates to a liquid preconcentrate composition comprising topiramate or a pharmaceutically acceptable addition salt thereof as active ingredient and an organic solvent selected from ethanol; glycerol; PEG such as for instance PEG 300, PEG 400, PEG 500 or PEG 600; propylene glycol or mixtures thereof, said preconcentrate composition having a low water content.

Preferably, the organic solvent is glycerol; PEG such as for instance PEG 300, PEG 400, PEG 500 or PEG 600, in particular PEG 300, PEG 400 or PEG 600, more in particular PEG 300 or PEG 400; propylene glycol or mixtures thereof, more preferably glycerol; PEG, in particular PEG 400; a mixture of glycerol with another organic solvent selected from PEG, in particular PEG 400, propylene glycol, or mixtures thereof, or a mixture of PEG, in particular PEG 400, with propylene glycol. More preferably, the organic solvent is a mixture of glycerol and PEG 400.

As already indicated hereinabove, the present composition comprises topiramate or a pharmaceutically acceptable addition salt thereof as active ingredient. Appropriate pharmaceutically acceptable addition salts of topiramate include those derived from pharmaceutically acceptable, inorganic and organic bases. Salts derived from appropriate bases include alkali metal (e.g. sodium, potassium), alkaline earth metal (e.g. magnesium), and choline. U.S. Pat. No. 6,559,293, and PCT International Applications Publication Nos. WO2003/070738 and WO2003/006467 describe the preparation and use of topiramate salts.

Basic salts of topiramate may increase the pH of the liquid composition upon reconstitution and this may have an effect on the antimicrobial activity of preservatives, which may be present in the composition. Salts may also interact with other components of the composition. Therefore, the active ingredient of the present composition is preferably topiramate free acid.

In a preferred embodiment of the present invention, a preconcentrate composition is provided which, upon dilution with an aqueous medium, results in a formulation from which the whole intended patient population can be dosed, i.e. babies and children ranging from 0 to about 6 years. Thus, preferably, the resulting formulation provides for dosing within a range of about 1 to about 10 mg of topiramate/kg. This implies that the reconstituted formulation preferably provides for accurate dosing of babies as well as for appropriate dosing with acceptable amounts of organic solvent for the older children of the intended patient population.

Therefore, the preconcentrate composition of the present invention preferably comprises topiramate or a pharmaceutically acceptable addition salt thereof in a concentration ranging from about 10 mg/ml to about 40 mg/ml (topiramate equivalent), more preferably ranging from about 15 mg/ml to about 40 mg/ml, even more preferably from about 20 mg/ml to about 40 mg/ml and most preferred is about 30 mg/ml.

The present invention also relates to a liquid composition, preferably a solution, obtainable by mixing the preconcentrate composition according to the present invention with an aqueous medium, preferably with water, more preferably with purified water. Preferably, said liquid composition provides for at least two doses. More preferably, the liquid composition provides for doses for 1 day to up to several weeks, e.g. up to 4 or 6 weeks. The fact that the preconcentrate is diluted ex tempore to a formulation from which at least two doses can be provided, implies that dosing can be easily adjusted based upon the weight of the patient. Dosing from a diluted formulation is more accurate than dosing from a non-diluted, more concentrated and hence often more viscous composition.

The liquid composition obtainable by diluting the preconcentrate composition is preferably suitable for oral administration.

The composition resulting from reconstitution of the preconcentrate composition with an aqueous medium preferably comprises topiramate or a pharmaceutically acceptable addition salt thereof in a concentration ranging from about 2.5 to about 10 mg/ml (topiramate equivalent), more preferably from about 5 to about 10 mg/ml and most preferred is about 5 mg/ml.

The shelf life of the present preconcentrate composition or of the liquid composition resulting from reconstitution of the preconcentrate with an aqueous medium, may be increased by the presence of one or more preservatives to prevent or retard growth of micro-organisms such as bacteria, yeasts and fungi in the formulation. Therefore, the preconcentrate composition of the present invention preferably comprises one or more preservatives. The organic solvent itself may not have a sufficient antimicrobial activity for the composition of the present invention or for the composition upon dilution or may not be active against certain micro-organisms.

When using a combination of preservatives, the quantities of these preservatives can be reduced as compared to the use of a single preservative, while retaining compliance with the requirements on microbial counts stipulated by the Pharmacopoeia.

Decreasing the concentration of the preservatives reduces the risk of undesired side-effects.

Pharmaceutically acceptable preservatives include quaternary ammonium salts such as benzalkonium chloride, alcohols such as benzyl alcohol, organic acids or salts and derivatives thereof such as benzoic acid, sodium benzoate, sorbic acid, potassium sorbate, propionic acid, sodium propionate, parabens such as methyl parahydroxybenzoate, propyl parahydroxybenzoate, ethyl parahydroxybenzoate or butyl parahydroxybenzoate, aqua conservans; chloorhexidine diacetate,-digluconate. Given the intended use of the present composition, the preservatives are preferably suitable for pediatric use. Preferred preservatives are parabens such as methyl parahydroxybenzoate, propyl parahydroxybenzoate, ethyl parahydroxybenzoate or butyl parahydroxybenzoate, in particular methyl parahydroxybenzoate or propyl parahydroxybenzoate.

The preservatives are present in the composition in a concentration in order to provide sufficient antimicrobial activity in the preconcentrate composition or in the liquid composition upon reconstitution. Preferably, the concentration of the preservatives in the resulting reconstituted liquid composition ranges up to about 3% (w/w), more preferably up to about 2.5% (w/w), more preferably up to about 2% (w/w), depending on the actual preservative being used.

The composition of the present invention may also contain one or more anti-oxidants, such as, for example, sodium metabisulfite, sodium bisulfite, sodium sulfite, sodium thiosulfate, ascorbic acid, BHA (butylhydroxyanisol), BHT (butylhydroxytoluene), vitamine E, propylgallate, ascorbyl palmitate, or complex forming agents such as EDTA (ethylenediaminetetraacetic acid), citric acid, tartaric acid, sodium-hexametaphosphate and the like. Given the intended use of the present composition, the antioxidants or the complex forming agents are preferably suitable for pediatric use. Preferred antioxidants are BHA, BHT, vitamine E or propylgallate.

The antioxidants or the complex forming agents are present in the present preconcentrate composition in a concentration in order to provide sufficient protection against oxidation in the preconcentrate composition or in the resulting liquid composition upon reconstitution. The concentration of the anti-oxidants generally amounts up to about 0.2% (w/v) and the amount of complex forming agents up to about 3% (w/v) of the preconcentrate composition or of the resulting liquid composition upon reconstitution.

In order to protect the present composition from degradation by the presence of O₂, the preconcentrate composition and/or the container containing the preconcentrate composition may also be dearated, for instance by degassing and working under inert atmosphere, for instance under Argon or N₂ atmosphere. This is a preferred embodiment of the present invention especially when the organic solvent comprises PEG or propylene glycol.

The preconcentrate composition is preferably filled in a container which is oversized in view of the volume of the composition itself in order to be able to accommodate an appropriate amount of an aqueous medium to reconstitute the composition. Preferably, the amount of O₂ in the container above the composition is about 7% or less.

The present invention therefore also relates to a container comprising an appropriate amount of the preconcentrate composition of the present invention. Said container can preferably accommodate at least twice the volume of the preconcentrate composition. More preferably, the container can accommodate 1 part of the preconcentrate composition and 5 parts of aqueous medium.

The preconcentrate composition of the present invention may also comprise pH adjusting agents in order to provide a pH value upon reconstitution wherein the antimicrobial activity of the preservatives can be maintained.

Preferably, the pH of the liquid composition upon reconstitution ranges from about 5 to about 8, more preferably from about 5.5 to about 7.5, most preferred is about 7.

As pH adjusting agent, there may be used buffer systems comprising mixtures of appropriate amounts of an acid such as phosphoric, succinic, tartaric, lactic, or citric acid, and a base, in particular sodium hydroxide, disodium hydrogen phosphate, sodium dihydrogen phosphate, sodium citrate and the like. Alternatively, the pH can also be adjusted by addition of an acid such as hydrochloric acid or a base such as sodium hydroxide and the like. In view of the fact that the pH adjusting agents have to be soluble in the preconcentrate composition comprising an organic solvent, acids or bases are preferred. In particular, bases are preferred, more in particular sodium hydroxide.

In order to accelerate the dissolution of the base, in particular sodium hydroxide, in the preconcentrate composition, and in order to prevent possible interaction between the base, in particular sodium hydroxide, and an organic solvent such as for example PEG, the base is preferably added to the present preconcentrate composition as an aqueous solution in a limited amount of water taken into account that the preconcentrate composition has a low water content, preferably that the amount of added water does not exceed about 5% by weight of the composition, more preferably does not exceed about 2.5% by weight, even more preferably does not exceed about 1% by weight or that the composition is substantially free of water, in view of the sensitivity of topiramate to hydrolysis in the presence of water.

The preconcentrate composition of the present invention has preferably a shelf life of about 2 years and the reconstituted composition can preferably be used for up to about 6 weeks. The reconstituted composition is preferably stored at low temperature, e.g. in the refrigerator.

In order to increase the palatability of the liquid composition upon reconstitution with an aqueous medium, sweetener(s) and/or flavour(s) may be added to the composition in order to mask the bitter taste of topiramate.

Suitable sweeteners include sucrose, glucose, fructose or intense sweeteners, i.e. agents with a high sweetening power when compared to sucrose (e.g. at least 10 times sweeter than sucrose). Suitable intense sweeteners comprise aspartame, saccharin, sodium or potassium or calcium saccharin, acesulfame potassium, sucralose, alitame, xylitol, cyclamate, neomate, neohesperidine dihydrochalcone or mixtures thereof, thaumatin, palatinit, stevioside, rebaudioside, Magnasweet®. The total concentration of the sweeteners may range from effectively zero to about 300 mg/ml based on the liquid composition upon reconstitution. Preferably, the sweetener is sucralose.

Suitable flavours include fruit flavours such as tutti frutti, cherry, raspberry, black currant or strawberry flavour, or stronger flavours, such as Caramel Chocolate flavour, caramel sweet tone, Mint Cool flavour, Fantasy flavour, vanilla, grenadine, guarana, masking flavour (Givaudan, in particular masking flavour 11031-31) and the like. Combinations of flavours may also be used. The total concentration of the flavouring substances may range up to about 0.5% (w/v), preferably from about 0.01% to about 0.5% (w/v), more preferably from about 0.03% to about 0.2% and most preferably from about 0.05% to about 0.15% based on the liquid composition upon reconstitution. Preferably, the present composition contains as flavouring agent a combination comprising grenadine and masking flavour, in particular masking flavour 11031-31 (Givaudan), or alternatively, the present composition contains as flavouring agent at least mint flavour.

In a preferred embodiment of the present invention, the composition contains both sweetener(s) and flavour(s).

Interesting compositions according to the present invention comprise the following:

| Topiramate or a pharmaceutically | about 10 mg to about 40 mg |
|---|---|
| acceptable addition salt thereof | (topiramate equivalent) |
| Organic solvent selected from ethanol, | ad 1 ml |
| glycerol, PEG, propylene glycol or | |
| mixtures thereof | |

| Topiramate or a pharmaceutically | about 10 mg to about 40 mg |
|---|---|
| acceptable addition salt thereof | (topiramate equivalent) |
| Preservative(s) | q.s. to give a concentration |
| | in the composition upon |
| | reconstitution ranging up |
| | to about 3% (w/w) |
| Organic solvent selected from ethanol, | ad 1 ml |
| glycerol, PEG, propylene glycol or | |
| mixtures thereof | |

| Topiramate or a pharmaceutically | about 10 mg to about 40 mg |
|---|---|
| acceptable addition salt thereof | (topiramate equivalent) |
| Preservative(s) | q.s. to give a concentration |
| | in the composition upon |
| | reconstitution ranging up |
| | to about 3% (w/w) |
| Sweetener(s) and/or flavour(s) | q.s. to give palatable |
| | composition upon |
| | reconstitution |
| Organic solvent selected from ethanol, | ad 1 ml |
| glycerol, PEG, propylene glycol or | |
| mixtures thereof | |

| Topiramate or a pharmaceutically | about 10 mg to about 40 mg |
|---|---|
| acceptable addition salt thereof | (topiramate equivalent) |
| Preservative(s) | q.s. to give a concentration |
| | in the composition upon |
| | reconstitution ranging |
| | up to about 3% (w/w) |
| Sweentener(s) and/or flavour(s) | q.s. to give palatable |
| | composition upon |
| | reconstitution |
| Acid or base | q.s. to give pH of about |
| | 5-8 (reflects pH of the |
| | composition upon |
| | reconstitution) |
| Organic solvent selected from ethanol, | ad 1 ml |
| glycerol, PEG, propylene glycol or | |
| mixtures thereof. | |

The present invention also relates to a process of preparing a preconcentrate composition of the present invention comprising the steps of:

dissolving topiramate or a pharmaceutically acceptable addition salt thereof and

optionally additional ingredients, in organic solvent while mixing, followed by adding organic solvent to the final volume.

More in particular, the present invention relates to a process of preparing a preconcentrate composition of the present invention comprising the steps of:

a) dissolving one or more preservatives in organic solvent;

b) dissolving topiramate or a pharmaceutically acceptable addition salt thereof in the solution of a);

c) dissolving sweetener(s) and/or flavour(s) in the solution of b);

d) adding organic solvent to the solution of c) up to the final volume;

e) optionally dissolving an appropriate amount of base in water followed by adding said solution to the solution obtained under d).

The above general route of preparing the composition of the present invention may be modified by a person skilled in the art by for instance adding certain ingredients at other stages than indicated above. For example, the sweetener(s) and/or flavour(s) can first be dissolved followed by dissolving the topiramate.

Experimental Part

Composition A:

| | |
|---|---|
| topiramate (free acid) | 30 mg |
| methyl parahydroxybenzoate 21.6 mg | |
| propyl parahydroxybenzoate | 2.4 mg |
| sucralose | 30 mg |
| grenadine flavour | 4.8 mg |
| masking flavour, in particular | |
| masking flavour 11031-31 (Givaudan) | 2.4 mg |
| sodium hydroxide | q.s. ad pH 7 (reflects pH |
| | value of the composition |
| | upon reconstitution) |
| purified water | 7.5 μl |
| polyethylene glycol 400 (PEG 400) | 500 mg |
| glycerol | q.s. ad 1000 μl |

Composition A for a 100 L Batch

| | |
|---|---|
| topiramate (free acid) | 3.0 kg |
| methyl parahydroxybenzoate | 2.16 kg |
| propyl parahydroxybenzoate | 0.24 kg |
| sucralose | 3.0 kg |
| grenadine flavour | 0.48 kg |
| masking flavour, in particular | |
| masking flavour 11031-31 (Givaudan) | 0.24 kg |
| sodium hydroxide | q.s. ad pH 7 (reflects |
| | pH value of the |
| | composition upon |
| | reconstitution) |
| purified water | 0.75 L |
| polyethylene glycol 400 (PEG 400) | 50.0 kg |
| glycerol | q.s. ad 100 L |

Synthesis of Composition A for a 100 L Batch

Polyethylene glycol 400 was charged to a suitable vessel. Methyl parahydroxybenzoate and propyl parahydroxybenzoate were added and the mixture was mixed until dissolution of the preservatives. Topiramate was added to the solution and the mixture was mixed until dissolution of topiramate. Sucralose was added to the solution followed by mixing. Grenadine flavour and masking flavour 11031-31 (Givaudan) were added and the mixture was mixed. Glycerol was added up to the final volume and the solution was mixed until homogeneous. Sodium hydroxide was dissolved in purified water and this solution was added to the vessel and the mixture was mixed. The solution was stirred under an inert atmosphere, preferably N₂. The solution was filtered (25 μm) and filled (15 ml) into glass bottles (100 ml). These steps were performed under inert atmosphere.

Compostion A is preferably diluted with purified water prior to administration in a ratio of 1 part of preconcentrate and 5 parts of purified water. The parts are preferably volume parts.

Therefore, before administration, the 100 ml bottles may be reconstituted up to 90 ml with water. This reconstitution is preferably performed by the pharmacist upon dispensing the oral pediatric topiramate formulation.

A further aspect of the present invention concerns the use of the preconcentrate composition or reconstituted preconcentrate composition as a medicine, especially the use thereof for the manufacture of a medicament for treating patients, in particular babies and children, suffering from a disease which is treatable by the administration of topiramate or a pharmaceutically acceptable addition salt thereof, such as seizures in patients with simple and complex partial epilepsy, primary or secondary generalized seizures and seizures associated with Lennox-Gastaut syndrome, migraine headaches. The present invention further relates to a method of treating patients, in particular babies and children, suffering from a disease which is treatable by the administration of topiramate or a pharmaceutically acceptable addition salt thereof, such as seizures in patients with simple and complex partial epilepsy, primary or secondary generalized seizures and seizures associated with Lennox-Gastaut syndrome, migraine headaches by administering to said babies and children a therapeutically effective amount of a composition or reconstituted preconcentrate composition of the present invention.

The daily required dosage of topiramate or a pharmaceutically acceptable addition salt thereof, the amount per single dose and the frequency of dosing varies with the condition being treated, the severity of said condition, and the patient being treated. Optimal dosages to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases and medications, may result in the need to adjust dosages.

While the foregoing specification teaches the principles of the present invention, with examples provided for the purpose of illustration, it will be understood that the practice of the invention encompasses all of the usual variations, adaptations and/or modifications as come within the scope of the following claims and their equivalents. It is also understood that whether the term “about” is used explicitly or not with quantitative expressions, every quantity given herein is meant to refer to the actual given value, and it is also meant to refer to the approximation to such given value that would reasonably be inferred based on the ordinary skill in the art, including approximations due to the experimental and/or measurement conditions for such given value.

1. A liquid preconcentrate composition comprising topiramate or a pharmaceutically acceptable addition salt thereof, as active ingredient and an organic solvent, said composition having a low water content.

2. A composition according to claim 1 wherein the composition is a solution.

3. A composition according to claim 1 wherein the organic solvent is selected from ethanol, glycerol, PEG, propylene glycol or mixtures thereof.

4. A composition according to claim 3 wherein the organic solvent is selected from glycerol, PEG, propylene glycol or mixtures thereof.

5. A composition according to claim 4 wherein the organic solvent is glycerol; PEG 400; a mixture of glycerol with another organic solvent selected from PEG 400, propylene glycol or a mixture thereof, or a mixture of PEG 400 with propylene glycol.

6. A composition according to claim 5 wherein the organic solvent is a mixture of glycerol and PEG 400.

7. A composition according to claim 1 wherein the active ingredient is topiramate free acid.

8. A composition according to claim 1 wherein the concentration of topiramate or a pharmaceutically acceptable addition salt thereof ranges from about 10 mg/ml to about 40 mg/ml (topiramate equivalent).

9. A composition according to claim 8 wherein the concentration of topiramate or a pharmaceutically acceptable addition salt thereof ranges from about 20 mg/ml to about 40 mg/ml (topiramate equivalent).

10. A composition according to claim 9 wherein the concentration of topiramate or a pharmaceutically acceptable addition salt thereof is about 30 mg/ml (topiramate equivalent).

11. A composition according to claim 1 further comprising one or more preservatives.

12. A composition according to claim 11 wherein the one or more preservatives are parabens.

13. A composition according to claim 1 further comprising one or more sweeteners and/or flavours.

14. A composition according to claim 1 further comprising a base.

15. A composition according to claim 1 wherein the water content is about 2.5% by weight or less.

16. A composition according to claim 15 wherein the water content is about 1% by weight or less.

17. A composition according to claim 1 having the following composition:

| | |
|---|---|
| topiramate (free acid) | 30 mg |
| methyl parahydroxybenzoate | 21.6 mg |
| propyl parahydroxybenzoate | 2.4 mg |
| sucralose | 30 mg |
| grenadine flavour | 4.8 mg |
| masking flavour 11031-31 | 2.4 mg |
| sodium hydroxide | q.s. ad pH 7 |
| | (reflects pH value of |
| | the composition upon |
| | reconstitution) |
| purified water | 7.5 μl |
| polyethylene glycol 400 | 500 mg |
| glycerol | q.s. ad 1000 μl |

18. A liquid composition for oral administration obtainable by mixing the composition according to claim 1 with an aqueous medium.

19. A liquid composition according to claim 18 wherein the composition is a solution.

20. A liquid composition according to claim 19 wherein the concentration of topiramate or a pharmaceutically acceptable addition salt thereof ranges from about 2.5 to about 10 mg/ml (topiramate equivalent).

21. A liquid composition according to claim 20 wherein the concentration of topiramate or a pharmaceutically acceptable addition salt thereof ranges from about 5 to about 10 mg/ml.

22. A liquid composition according to claim 21 wherein the concentration of topiramate or a pharmaceutically acceptable addition salt thereof is about 5 mg/ml.

23. A liquid composition according to claim 18 comprising about 1 part of a composition according to claim 17 and about 5 parts of an aqueous medium.

24. A method for treating a disorder selected from the group consisting of seizures in patients with simple and complex partial epilepsy, primary or secondary generalized seizures, seizures associated with Lennox-Gastaut syndrome, and migraine headaches comprising administering to a patient in need thereof a liquid composition according to claim 18.

25. A container comprising the composition according to claim 1.

26. A container according to claim 26 comprising about 15 ml of the composition according to claim 17.

27. A container according to claim 27 wherein the container can accommodate about 100 ml.
